(19) United States
(12) Patent Application Publication
(10) Pub. No.: US 2002/0115126 A1
(43) Pub. Date: Aug. 22, 2002

(54) Cholesterol desaturases from ciliates, methods and uses

(76) Inventor: German A. Valcarce, Buenos Aires (AR)

Correspondence Address: BRUCE LONDA, NORRIS, MCLAUGHLIN & MARCUS, P.A., 220 EAST 42ND STREET, 30TH FLOOR, NEW YORK, NY, 10017, US

(21) Appl. No.: 10/074,116

(22) Filed: Feb. 12, 2002

Related U.S. Application Data
(63) Continuation-in-part of application No. 09/835,804, filed on Apr. 16, 2001.
Application No. 09/835,804 is a continuation-in-part of application No. 09/641,609, filed on Aug. 17, 2000.
(60) Provisional application No. 60/153,754, filed on Sep. 13, 1999.
(60) Provisional application No. 60/153,741, filed on Sep. 13, 1999.
(60) Provisional application No. 60/172,844, filed on Dec. 20, 1999.
(60) Provisional application No. 60/177,252, filed on Jan. 20, 2000.

(51) Int. Cl. C12Q 1/60

(52) U.S. Cl. 435/11

(57) ABSTRACT

A cell free extract from a ciliate microorganism, including protozoo Tetrahymena, wherein the extract comprises a homogenate a microsomal fraction, a desaturase-enriched fraction or a mixture thereof and, more particularly the invention provides substantially pure enzymes: 7 cholesterol desaturases and 22 cholesterol desaturases. The invention also relates to methods for recovering cholesterol desaturases activities from ciliates, including Tetrahymena, and their use in a production of Vitamin D precursors and analogs from several cholesterol sources.

### FIELD OF THE INVENTION

The present invention relates to a novel cholesterol desaturases from Ciliata phylum microorganism as for example Tetrahymena, and also relates to a process for production of these enzymes. More particularly, the invention relates to methods for recovering of 7 cholesterol desaturase and 22 cholesterol desaturase from cell free extracts, including homogenate fractions, microsomal fractions, desaturase enriched fractions and/or mixtures thereof.

The invention also relates methods for treating cholesterol-containing substrates for the production of Vitamin D precursors and analogs, in particular 7 dehydrocholesterol and 7,22 bis dehydrocholesterol from pure cholesterol, cholesterol-enriched preparations or cholesterol-containing products.

### BACKGROUND OF THE INVENTION

The increased blood cholesterol concentration in humans seems to have a direct positive correlation with coronary heart disease. It is widely recognized that a main contributor to high levels of blood cholesterol is the kind of diet. Consumption of foods containing high fat and cholesterol levels contributes significantly to increase the rates of heart diseases. Patients with coronary heart disease or hypercholesterolemia, therefore, are commonly recommended to reduce their dietary cholesterol intake.

Foodstuffs of animal origin such as eggs and milk are commonly used in the preparation of a variety of food products. Because of the special organoleptic traits of milk and eggs, it is difficult to replace them by other products with less cholesterol content.

Accordingly, there is a continuing and real interest in decreasing the intake of food substances that have high cholesterol content.

A number of methods have been disclosed and employed for reducing the cholesterol content of foodstuffs. It is known that food with reduced cholesterol contents may be obtained by physical and chemical methods, for example by extracting cholesterol from food with liquid dimethylether (Yano et al, 1980, U.S. Pat. No. 4,234,619) or other organic solvents Johnson et al. (U.S. Pat. No. 4,997,668, Fallis et al, 1978, U.S. Pat. No. 4,104,286).

A variation on the use of organic solvents is to employ oils to extract cholesterol in egg and dairy products, (Bracco et al, 1982, U.S. Pat. No. 4,333,959; Keen, 1991, U.S. Pat. No. 5,039,541; Conte et al, 1992, U.S. Pat. No. 5,091,203; Merchant et al, 1995, U.S. Pat. No. 5,378,487; Jackeschky, 1998, U.S. Pat. No. 5,780,095).

In the physical methods described above, fats, proteins, pigments and flavors components are also extracted, in addition to cholesterol, causing deterioration of the treated foodstuff quality. Again, these methods do not selectively extract cholesterol, and products contaminated with oils are inevitably produced, which is undesirable.

Other prior art methods are well known such as:

The use of supercritical fluids extraction of egg products (Ogasahara et al, U.S. Pat. No. 5,116,628, 1992).

The production of low cholesterol butter oil by vapor sparging, disclosed by Conte et al (1992, U.S. Pat. No. 5,092,964).

However, the extreme conditions necessary for carrying out the process and the high cost involved therein are important drawbacks for the application of this method.

Removal of cholesterol by formation of complexes with cyclodextrins has also been proposed for fatty substances of animal origin (Courregelongue et al, 1989, U.S. Pat. No. 4,880,573) and specifically in the case of dairy products (Chung Dae-Won, 1999, Foreign Patent WO 9917620). The formation of complexes of cholesterol and saponin has also been disclosed as a means to reduce cholesterol in milk (Richardson, 1994, U.S. Pat. No. 5,326,579). These methods are, however, too expensive for industrial applications.

A different approach is based on the use of enzymes that modify cholesterol. Thus, the use of cholesterol reductases, that modify cholesterol into poorly absorbed sterols, has been proposed (Beitz et al, 1990, U.S. Pat. No. 4,921,710; Ambrosius et al, 1999, U.S. Pat. No. 5,856,156). Another proposed enzymatic approach is the conversion of cholesterol into epicholesterol, which is then further modified by an epicholesterol dehydrogenase (Saito et al, 1999, U.S. Pat. No. 5,876,993). These methods are disadvantageous in that they do not result in the conversion of cholesterol into useful compounds for human nutrition.

There is therefore a need of methods for treating foodstuffs for reducing the amount of cholesterol. Preferably, the cholesterol is converted into one or more substances that are useful for human nutrition.

Vitamin D3, also named Cholecalciferol, is commonly prepared, in commercial practice, by irradiation of 7-dehydrocholesterol with ultraviolet light. Upon irradiation, intermediate products are formed (pre vitamin D3) which are converted into vitamin D3 by thermal rearrangement.

The precursor 7- dehydrocholesterol may be obtained via organic solvent extraction of animal skin (cow, pig, sheep) followed by extensive purification and crystallization.

Industrially, 7 dehydrocholesterol is commonly synthesized from cholesterol via a laborious chemical synthesis entailing the introduction of a double bond in the seven position of cholesterol. However, the introduction of this double bond in the seven position of cholesterol has several associated problems. (Gui-Dong Zhu and William Okamura, 1995, Chemical Reviews 95: 1877-1952).

The usual methods of synthesis from compounds having a 5-steroid structure, consist in the classical bromination/dehydrobromination procedure using 1,3 dibromo-5,5 dimethylhydantoin or N-bromosuccinimide. The resulting epimeric mixture of C-7 bromides is treated with trimethyl phosphite in xylene or collidine, yielding about 40% of the desired 5,7 diene, along with a substantial quantity of the undesired 4,6-diene isomer (25%).

The unwanted contaminant, delta 4,6 diene, has plagued this conversion despite many decades of extensive studies, and several improvements for this process have been reported. (Confalone c.s. in J. Org. Chem. 1981, 46, 1030-32; German Patent Application No. 2547199).

For instance, the dehydrobromination of the 7-alfa-bromide of the acetate of cholesterol with Bu₄NF.H₂O in THF or DMF was reported to give selectively the desired delta 5,7 diene. In this two-step procedure, provitamin D synthesis was achieved in 62% yield with 95% purity. Another possible alternative is the palladium catalyzed elimination of allylic esters, which yields 90% of the desired homoannular diene.

Because the introduction of the delta 7,8 double bond is cumbersome, another approach has been to generate the desired 5,7 diene through base-catalyzed deconjugation of the easily available 1,4,6 trien-3-one. This reaction is followed by succesive reduction, diene protection and epoxidation. This simple route is hampered, however, by the low selectivity of the epoxidation step.

In conclusion, although many solutions have been proposed for improving the chemical synthesis of provitamin D, the great number of reaction steps and the complexity of the synthesis constitute a serious disadvantage. Moreover, yields in these syntheses are usually poor, chemicals needed for carrying out the procedures may have to be specially prepared, and in some cases also special equipment is required.

A better possibility of synthesizing these biologically active compounds from cholesterol is therefore of great importance.

Selective introduction of a double bond on the seven position of a 5 sterol is important also for the synthesis of intermediates for important products, i.e, in the preparation of 1,25 dihydroxy vitamin D derivatives.

In addition, due to the toxicity of the required chemicals, an environmentally friendly process for the synthesis of provitamin D and its analogs is desired.

In accordance with the foregoing and for the purpose of providing low-cholesterol foods fortified with useful compounds, enzymes for selective transformation of cholesterol to one or more substances that are useful for human nutrition are in demand.

In addition, simple and safe processes for producing unsaturated derivatives such as: Δ7 dehydrocholesterol (also named provitamn D3), Δ22 dehydrocholesterol and Δ7,22 bis dehydrocholesterol (a close analog of pro vitamin D2) having a high purity is also desired.

It is known that live Tetrahymena is capable of converting cholesterol into more unsaturated derivatives such as: Δ7 dehydrocholesterol (also named provitamn D3), Δ22 dehydrocholesterol and Δ7,22 bis dehydrocholesterol (a close analog of pro vitamin D2) during growth in cholesterol containing media (Conner, R. L., et al., (1969) J Biol Chem 244:2325-2333; Mallory, F. B. and Conner, R. L. (1971) Lipids 6:149-153; Conner, R. L., et al., (1978) Lipids 13: 692-696; Ferguson, K. A., et al., (1975) J Biol Chem 250:6998-7005).

The desaturation of cholesterol at position 7 converts the same into provitamin D3 and pro vit D derivatives, which upon UV irradiation can be activated into vitamin D. This biotransformation has seldom been observed in nature.

Furthermore, the mechanism of these reactions have been partially disclosed (Zander and Caspi).

Moreover, the biological system capable of performing cholesterol desaturations is not known: therefore, the attempts to fully explain the reaction mechanism have been frustrated.

The discovery of enzymes for converting cholesterol in vitamin D derivatives is desirable, because it is to be expected that it might be useful for:

1. The reduction of cholesterol in foodstuff with simultaneous increasing of more unsaturated derivatives.

2. The production of pro vitamin D and pro vitamin D analogs from cholesterol

### SUMMARY OF THE INVENTION

It is therefore an object of the present invention to provide a cell free extract and substantially pure enzymes useful for the process of reducing cholesterol in foodstuffs and increase their content in provitamin D.

It is still another object of the present invention to provide a cell free extract and substantially pure enzymes useful for the process of synthesis for provitamin D and its analogs from cholesterol substrates.

It is a further object of the present invention to provide a cell free extract from ciliates, more specifically from Tetrahymenidae family microorganism, wherein said cell free extract contains cholesterol desaturase activities selected from the group comprising Δ7 and Δ22 cholesterol desaturases activities that catalyze desaturation of cholesterol.

The cell free extract is preferably selected from the group consisting of cell free homogenate, microsomal fraction and desaturase-enriched fraction, or a combination thereof, all from ciliates, more specifically from Tetrahymenidae family microorganism.

It is still another object of the present invention to provide a process for manufacturing 7 dehydrochlesterol (provitamin D3) and Δ7,22 dehydrocholesterol comprising:

(a) mixing a cell free extract of the invention with a cholesterol containing substrate;

(b) incubating the mixture for a period of time enough to produce Δ7 dehydrocholesterol and Δ7,22 bis dehydrocholesterol;

(c) recovering said Δ7 dehydrocholesterol and Δ7,22 bis dehydrocholesterol by solvent extraction and chromatographic purification.

It is another object of the present invention to provides a substantial pure Δ7 cholesterol desaturase enzyme from ciliates, more particularly from Tetrahymenidae family microorganism, wherein said enzyme is capable of catalyzing the conversion of a cholesterol substrate in 7 dehydrocholesterol by introducing a double bound at the position seven in the cholesterol molecule.

According to the invention, the substantial pure 7 cholesterol desaturase enzyme is characterized by

(a) having a molecular weight of approximately 60 kDa by gel chromatography;

(b) having an optimum pH range for enzymatic activity between 6.5-8.5;

(c) having an optimum temperature range for enzymatic activity of 28° C. to 35° C.;

(d) being unaffected by metal ions such as Ca⁺², Mn⁺² and Mg⁺²,EDTA concentrations and 2-mercaptoethanol;

(e) being inactivated after 1 minute at 100° C.;

(f) being storage at −20° C. by at least 6 months.

It is another object of the present invention to provides a substantial pure Δ22 cholesterol desaturase enzyme from ciliates, more particularly from Tetrahymenidae family microorganism, wherein said enzyme is capable of catalyzing the conversion of a cholesterol containing substrate in Δ22 dehydrocholesterol by introducing a double bound at the position twenty-two in the cholesterol molecule.

According to the invention, the substantial pure Δ22 cholesterol desaturase enzyme is characterized by

(a) having a molecular weight of approximately 60 kDa by gel chromatography;

(b) having an optimum pH range for enzymatic activity between 5.5-8.5;

(c) having an optimum temperature range for enzymatic activity of 28° C. to 35° C.;

(d) being unaffected by metal ions such as Ca⁺², Mn⁺² and Mg⁺² and EDTA concentrations;

(e) being inactivated after 1 minute at 100° C.;

(f) being storage at −20° C. by at least 6 months.

It is still a further object of the present invention to provide a process for preparing a substantial pure Δ7 cholesterol desaturase enzyme from ciliates, more specifically from Tetrahymenidae family, said process comprising the steps of:

(a) culturing a microorganism in a suitable medium, wherein said microorganism is capable of producing 7 cholesterol desaturases;

(b) Disintegrating the culture and extracting the same with buffer solution containing, if necessary, non-ionic surfactant or glycerol as stabilizer;

(c) subjecting the extract to a chromatography purification under suitable conditions; and

(d) eluting and recovering said 7 cholesterol desaturases.

It is still another object of the present invention to provide a process for preparing a substantial pure Δ22 cholesterol desaturase enzyme from ciliates, more specifically from Tetrahymenidae family microorganism, said process comprising the steps of:

(a) culturing a microorganism in a suitable medium, wherein said microorganism is capable of producing 22 cholesterol desaturases;

(b) disintegrating the culture and extracting the same with buffer solution containing, if necessary, non ionic surfactant or stabilizer as glycerol;

(c) subjecting the extract to a chromatography purification under suitable conditions; and

(d) eluting and recovering said 22 cholesterol desaturases.

It is another object of the present invention provides the use of substantial pure Δ7 cholesterol desaturase enzyme from ciliates, more specifically from Tetrahymenidae family microorganism to produce Δ7 dehydrocholesterol (provitamin D3) employing cholesterol containing substrates.

It is another object of the present invention provides the use of substantial pure Δ7 cholesterol desaturase and pure Δ22 cholesterol desaturase enzymes from ciliates, e.g. from Tetrahymenidae family microorganism, to produce Δ7,22 bis dehydrocholesterol employing cholesterol as substrate.

It is still another object of the present invention to provide compounds obtained by the method of the present invention which compounds constitute high value added compounds as they are synthetic intermediates in the preparation of vitamin D or vitamin D analogs.

The above and other objects, features and advantages of this invention will be better understood when taken in connection with the accompanying drawings and description.

### BRIEF DESCRIPTION OF THE DRAWINGS

FIG. 1 shows a chromatographic diagram corresponding to HIC of purification of Δ7 cholesterol desaturase. The solubized fraction from Tetrahymena membranes was applied to a Hydrophobic Interaction Chromatography column equilibrated and eluted. Unbonded protein fraction was collected and further purified by Ion Exchange Chromatography

FIG. 2 shows a chromatographic diagram corresponding to ionic exchange of purification of Δ7 cholesterol desaturase. The Δ7 cholesterol desaturase obtained from H.I.C was applied to a SP Sepharose HP column and eluted. The fraction was collected. Active fraction, indicated by an arrow, were pooled and further purified by Gel Filtration Chromatography.

FIG. 3 shows a chromatographic diagram corresponding to molecular exclusion of purification of Δ7 cholesterol desaturase. The Δ7 cholesterol desaturase obtained from the Ion Exchange step was applied to Suprose column and eluted. Fraction of 1.0 ml was collected and Δ7 cholesterol desaturase activity was assayed.

### DETAILED DESCRIPTION OF THE PREFERRED EMBODIMENT

The methods of the present invention provides several advantages. For example the method selectively transforms cholesterol to provitamin D3. During the process, no other isomers are formed in addition to 7 dehydrocholesterol, avoiding the complex problem associated with the purification of the product from sterol mixtures.

The method selectively transforms cholesterol from sterol mixtures avoiding the drawbacks associated with high purification of the starting material.

The method also has a high yield and avoid the use of toxic products and complex compounds used in the current chemical method of synthesis of 7 dehydrocholesterol.

By employing the cell free extracts and substantially pure enzymes according to the invention, pro-vitamin D and their analogs may be obtained, under mild conditions, few steps and by a meted that is not contaminant because of their enzymatic and non chemical nature.

The term “cholesterol” comprises an aqueous suspension of cholesterol, an aqueous micelle solution of cholesterol, an aqueous solution in which cholesterol containing organic solvent layer is emulsified.

The term “cholesterol-containing product” comprises any animal product that may provide a rich source of cholesterol, i.e., skin, feathers, eggs, etc.

Preparation of a protozoan culture: any suitable growth medium can be used for culturing the cells. For example, media based on yeast extract, glucose and skimmed milk can yield high cell densities at very low costs (Kiy, T. and Tiedtke, A. (1992), Appl. Microbiol. Biotechnol. 37:576-579, incorporated herein by reference). It is possible also to use a modification of the medium described by Kiy and Tiedtke. The composition of this medium includes yeast extract (0.1 to 10% w/v, preferably 0.5% w/v), glucose (0.1 to 5% w/v, preferably 1% w/v), iron citrate (0.0001 to 0.1% w/v, preferably 0.003% w/v), supplemented with between 1 and 50% v/v fluid skimmed milk (preferably between 5 and 20% v/v).

Cultivation of the protozoa useful in the practice of the invention can be achieved by any suitable means. For example, fermentors that utilize mechanical stirring, or air lift fermentors can be utilized for large-scale cultivation of the protozoa useful in the practice of the invention.

The optimal culture temperature depends on the strain of protozoa, but typically falls within the range of from 20° C. to 38° C. Similarly, the optimal culture pH depends on the strain of protozoa, but typically falls within the range of from pH 5.0 to pH 8.0. For example, a pH of 6.8 has been successfully used to cultivate Tetrahymena thermophila wild type strain CU399. Culture time also depends on the protozoan strain and the chosen method of cultivation. Typically, culture times of between 10 and 80 hours (such as 23-25 hours) are adequate.

Cultivation of the protozoa can be carried out with or without agitation. When agitation is used, exemplary rates of stirring are in the range of from 50 rpm to 600 rpm, typically about 100 rpm. Finally, to achieve high protozoan densities in a relatively short time, cultivation can be carried out in airlift bioreactors (Hellenbroich, D., et al., Appl. Microbiol. Biotechnol. 51:447-455 (1999), incorporated herein as reference) utilizing any suitable medium, such as the media disclosed in Tiedtke and Kiy (German Patent DE 4238842 incorporated herein as reference). Typically, cell densities up to 2×10⁷ cells/ml can be achieved.

This preferred embodiment of the invention is based upon the discovery that, by a very special manipulation of the sterol composition in the culture medium, the cholesterol desaturase content of the microorganisms is greatly augmented. Furthermore, selective provitamin D or analogs is obtained

Table 1 shows the effect of different growth conditions on desaturases productivity in Tetrahymena strain CU399. The activity is expressed as percentage of added cholesterol transformed by the solubilized fraction.

TABLE 1

| Sterol composition of | Δ 7 desaturase | Δ 22 desaturase |
|---|---|---|
| the medium | activity | activity |
| Without sterols | — | — |
| Add 1 mg/100 ml of | 2% | 30% |
| cholesterol | | |
| Add 1 mg/100 ml of 22 | 25% | 1% |
| dehydrocholesterol | | |

For commercial production of cholesterol desaturase the gene or genes for cholesterol desaturase may be transferred into another microorganism such as E. coli. The process would be similar to that now in use for making human insulin, bovine growth hormone, and porcine growth hormone.

Any microorganism including variants and mutants can be used for the production of the cholesterol desaturases, so long as it belongs to the genus Tetrahymena and is capable of producing cholesterol desaturases. A specific example of such microorganism is Tetrahymena CU399 and can be obtained from public depositories such as the American Type Culture Collection (ATCC), 10801 University Boulevard, Manassas, Va. 20110-2209, U.S.A.

It will be apparaent to any person skilled in the art that ciliate organism other than the Tetrahymena thermophyla may be employed for obtaining 7 cholesterol desaturase and 22 cholesterol desaturase enzymes. Such other ciliates are, for example: Tetrahymenidae as T. pyriformis, T. patula, T. rostrata, T. vorax, T. paravorax, T. chironomi, T. setifera, T. corlissi, T. stegomyae and T. limacis, and also from other family members of class Oligohymenophorea as Colpidium; and from members of class Nassophorea, such as Paramecium.

Assay of Cholesterol desaturase activity: the desaturase activity in the different fractions such as homogenate, microsomes, desaturase- enriched fractions or pure enzyme were tested by adding radio-labeled cholesterol, and various cofactors such as ATP, NAD, NADP or their reduced derivatives. After incubation at 30° C. during 1 to 6 hours, the reaction was stopped and the sterols were extracted from the reaction mixture and analyzed as described in Example 1.

The radioactive sterols present in the mixture were identified as cholesterol, Δ7-dehydrocholesterol, Δ22-dehydrocholesterol and Δ7-22didehydrocholesterol by co-migration in different chromatographic systems and mass spectroscopy analysis.

Characterization and Purification of Δ7 cholesterol desaturases:

A number of conditions were assayed for improving enzyme yields and for optimizing the activities measurements.

A significant increase in the desaturase activity (5 to 10 fold) was observed when Tetrahymena is grown in PPYE medium plus Δ22 dehydrocholesterol.

The 7 cholesterol desaturase from Tetrahymena of the present invention is a novel enzyme, which catalyze the reaction of cholesterol in accordance with the following equation:

Cholesterol+NAD(P)H→7 dehydrocholesterol+NAD(P)

The activity of the enzyme can easily be determined through the use of the above reaction, for example by determining the 7 dehydrocholesterol formed using a classical RP-HPLC method.

The physicochemical properties of the 7 cholesterol desaturase are as follows:

Optimal pH: The desaturase activity show optimal conversion rate at pH 7.4. Beyond the pH limits of 5 and 9.5, the desaturase is not active.

Temperature stability: optimal desaturase activity is obtained when the enzyme is incubated at temperatures from 30 to 35° C. Desaturase activity is sub optimal at temperatures below 25° C. and decrease when the enzyme incubation is carried out at temperatures upper than 42° C.

Detergent inhibition: the desaturase activity is affected by various detergents such as: triton X100 and X114, Tween 80 and deoxycholate. At concentration near to critical micellar concentration Octylthyoglucoside (OTG) effectively solubilize the enzyme and this concentration is only partially inhibitory for the enzyme.

Molecular weight: apparent molecular mass of the purified 7 cholesterol desaturase is estimated to be 60 kDa by gel filtration chromatography.

Ions and quelators effect: the desaturase rate is not influenced by EDTA concentration suggesting that the enzyme does not require divalent metals cations for activity. Effectively, the activity is not affected by Ca⁺², Mn⁺² and Mg⁺².

Storage conditions: Solubilized enzyme could be stored at −80° C. and −20° C. for at least 6 months with small change in enzymatic activity. The addition of 20% of glycerol stabilized the activity.

Lyophilization could also be used for storage. The use of 1 mg/ml of BSA showed optimal performance.

Specific activity: the rate of reaction of the purified enzyme is linear with respect to the enzyme protein content up to 5 mg/ml and the incubation time within 60 min.

Inhibitors: The activity was inhibited by the addition of Δ22 dehydrocholesterol to the reaction media.

The purification of 7 cholesterol desaturase may be carried out by employing any purification method known in the art, according to a preferred embodiment, however, the purification of 7 cholesterol desaturase is carried out as it is disclosed in Example 4. Following Table 2 shows the results of the specific activity and yielding when the purification method disclosed in Example 4 is employed.

TABLE 2

| | | | Specific | |
|---|---|---|---|---|
| | | | activity | |
| | Total | Total | (nmol/mg | |
| Purification | protein | activity | protein × | |
| step | (mg) | (nmol) | min) | Yield (%) |
| Microsomal | 5000 | 66000 | 0.11 | 100 |
| fraction | | | | |
| Soluble | 2800 | 43680 | 0.13 | 66 |
| fraction | | | | |
| (OTG) | | | | |
| Salt | 2200 | 39600 | 0.15 | 60 |
| precipitation | | | | |
| HIC | 272 | 34320 | 1.05 | 52 |
| IEC | 13.7 | 11880 | 7.20 | 18 |
| SEC | 2.9 | 5280 | 15.1 | 8 |

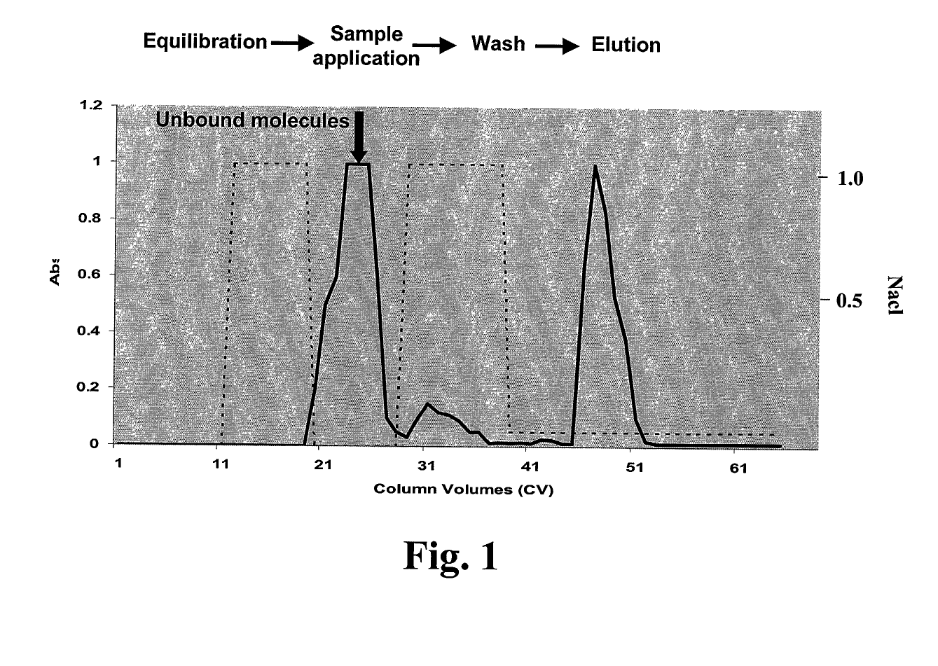

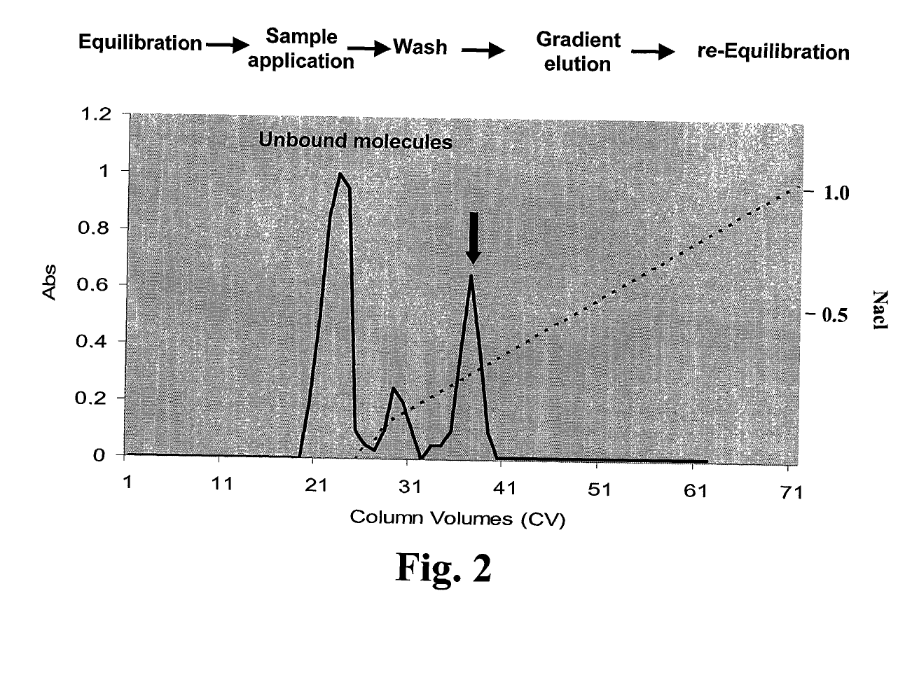

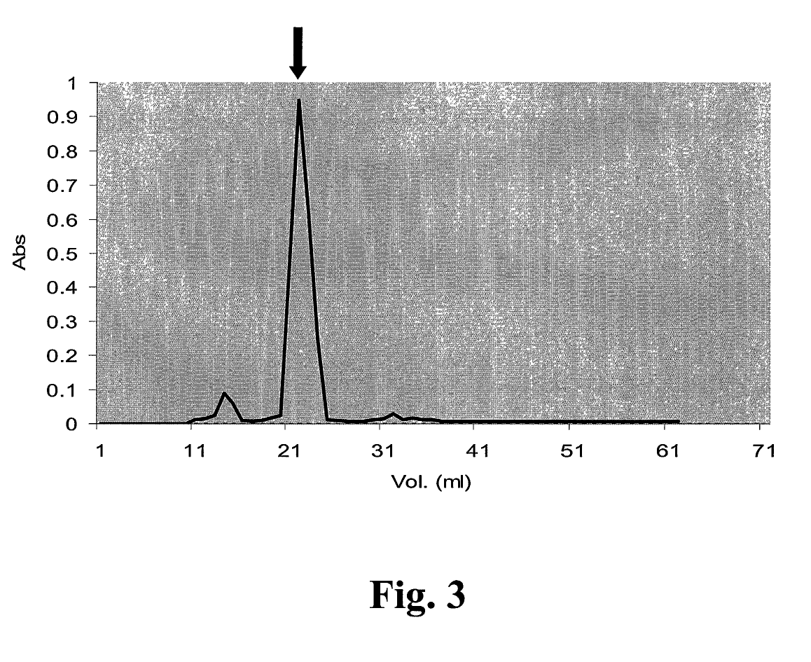

FIGS. 1, 2 and 3 show the chromatographic diagrams of purification of 7 cholesterol desaturase by a hydrophobic interaction chromatography, a cationic exchange chromatography and a molecular exclusion chromatography, respectively.

The substantially purified 7 cholesterol desaturase enzyme according to the invention is employed for obtaining 7 dehydrocholesterol (pro-vitamin D3), useful as an intermediate for obtaining Vitamin D according to methods well known in the art. Table 3 shows results from the cholesterol transformation into 7 dehydrocholesterol according to the method disclosed in Example 6. The reaction characteristics are listed in following Table 3.

TABLE 3

| | |
|---|---|
| Protein concentration | 1 mg/ml |
| Specific activity | 7 nmol/min × mg |
| (IEC fraction) | protein |
| Reaction time | 60 min |
| Conversion yield | 84% |
| Initial cholesterol | 0.5 mM |

By employing the method disclosed in Example 6 a high (84%) yield in the conversion of cholesterol into 7 dehydrocholesterol is obtained. Briefly, in one preferred embodiment of the invention, an enzymatic method for producing 7 dehydrocholesterol is provided which comprises the steps of: a) adding soluble cholesterol to the enzyme preparation selected from the group of: microsomal fraction, desaturase-enriched preparation containing Δ7 desaturase and pure Δ7 desaturase; b) incubating for a time enough for producing a high yield (above 85%) of 7 dehydrocholesterol; c) extracting the sterols with a water immiscible solvent d) recovering pure 7 dehydrocholesterol.

Characterization and Purification of Δ22 cholesterol desaturases:

The 22 cholesterol desaturase enzyme of the present invention is a novel enzyme, which catalyzes the reaction of cholesterol in accordance with the following equation:

Cholesterol+NAD(P)H→22 dehydrocholesterol+NAD(P)

The activity of the enzyme can easily be determined through the use of the above reaction, for example by determining the Δ22 dehydrocholesterol formed using a classical RP-HPLC method.

The physicochemical properties of 22 cholesterol desaturase are as follows:

Optimal pH: The desaturase activity show optimal conversion rate at pH 7.4. Beyond the pH limits of 5 and 9.5, the desaturase is not active.

Temperature stability: optimal desaturase activity was obtained when the enzyme is incubated at temperatures from 30 to 35° C. Desaturase activity is sub optimal at temperatures below 25° C. and decrease when the enzyme incubation is carried out at temperatures upper than 42° C.

Detergent inhibition: the desaturase activity is affected by various detergents such as: triton X100 and X114, Tween 80 and deoxycholate. At concentration near to critical micellar concentration Octylthyoglucoside (OTG) effectively solubilize the enzyme and this concentration is only partially inhibitory for the enzyme.

Molecular weight: apparent molecular mass of the purified 22 cholesterol desaturase is estimated to be 60 kDa by gel filtration chromatography.

Ions and quelators effect: the desaturase rate is not influenced by EDTA concentration suggesting that the enzyme does not require divalent metals cations for activity. Effectively, the activity is not affected by Ca⁺², Mn⁺² and Mg⁺².

Storage conditions: Solubilized enzyme could be stored at −80° C. and −20° C. for at least 6 months with small change in enzymatic activity. The addition of 20% of glycerol stabilized the activity.

Lyophilization could also be used for storage. The use of 1 mg/ml of BSA showed optimal performance.

Specific activity: the reaction velocity of the purified enzyme is linear with respect to the enzyme protein content up to 5 mg/ml and the incubation time within 120 min.

Inhibitors: The activity was inhibited by 2-mercaptoethanol.

The purification of 22 cholesterol desaturase may be carried out any purification method web known in the art; however, according to a preferred embodiment of the invention the purification of 22 cholesterol desaturase is carried out with the method disclosed in Example 5. Following Table 5 shows the results of the specific activity and yield when the purification method disclosed in Example 5 is employed.

TABLE 4

| | | | Specific | |
|---|---|---|---|---|
| | Total | Total | activity | |
| Purification | protein | activity | (nmol/mg | Yield |
| step | (mg) | (nmol) | protein × min) | (%) |
| Microsomal | 5000 | 85000 | 0.14 | 100 |
| fraction | | | | |
| Soluble | 2917 | 59500 | 0.17 | 70 |
| fraction | | | | |
| (OTG) | | | | |
| Salt | 2423 | 55250 | 0.19 | 65 |
| precipitation | | | | |
| IEC (anionic) | 55.8 | 22100 | 3.3 | 26 |
| SEC | 4.8 | 9350 | 16.0 | 11 |

The enzymes preparation (containing 7 cholesterol desaturase and/or 22 cholesterol desaturase) having the above-mentioned activities include, for example, purified enzymes, crude enzymes (such as homogenate, microsomal fraction and desaturase enriched fraction), and a treated matter of microbial cells as Tetrahymena having those enzyme activities.

Both desaturases enzymes of the present invention may be used in aqueous solutions or as a powder. Preferably, they are dissolved in water and the resulting aqueous solution is used in the different application. If necessary, surfactants such as OTG may be added to the solution. Other stabilizers such as polyols, glycerol, methanol can be used.

In accordance with the invention the enzymes can also be used immobilized to a solid matrix.

If necessary, coenzymes such as NAD(P), NAD(P)H may be added to the enzymes reaction.

The enzymes preparation (containing 7 cholesterol desaturase and 22 cholesterol desaturase) provides the obtention of another Vitamin D derivative, namely 7,22 bis-dehydrocholesterol by employing cholesterol as substrate.

It will be obvious for any person skilled in the art that the enzymes preparations (containing 7 cholesterol desaturase and/or 22 cholesterol desaturase) may be employed for obtaining several Vitamin D derivatives by employing cholesterol as substrate, with said cholesterol being in a pure form or forming part of several foods or other cholesterol sources, e.g. eggs, skin, feathers, milk, etc.

Table 5 shows the results of the transformation of cholesterol into 7,22 bis dehydrocholesterol according to the method disclosed in Example 7. The reaction characteristics are listed in following Table 5.

TABLE 5

| | |
|---|---|
| Protein concentration | 2 mg/ml |
| 7 specific activity | 3.5 nmol/min × |
| (IEC fraction) | mg protein |
| 22 specific activity | 3.3 nmol/min × |
| (IEC fraction) | mg protein |
| Reaction time | 60 min |
| Conversion yield | 84% |
| Initial cholesterol | 0.5 mM |

By employing the method disclosed in Example 7 a high (84%) yield in the conversion of cholesterol into 7,22 bis dehydrocholesterol is obtained. Briefly, in one preferred embodiment of the invention, an enzymatic method to produce 7,22 bis dehydrocholesterol is provided which comprise the steps of: a) adding soluble cholesterol to the 7 and 22 enzyme preparations, selected from the group of: microsomal fraction, desaturase- enriched preparation containing 7 desaturase and pure 7 desaturase. b) incubating for a sufficient time to produce 7,22 bis dehydrocholesterol with high yield (more than 85%) c) extracting the sterols with a water inmiscible solvent d) recover pure 7,22 bis dehydrocholesterol.

It is possible to increase the efficiency of this transformation by increasing the solubility of cholesterol, using detergents or emulsifiers, i.e, tween 80, arabic gum, triton X100, etc.

All the publications herein contained have been provided as references.

### EXAMPLES

### Example 1

### Preparation of the Homogenate Fraction

Cells and cultures: Tetrahymena thermophila wild type strain CU399 was maintained in axenic cultures. Inocula were prepared in medium containing 1% proteose peptone, 0.1% yeast extract (both from Difco Laboratories, Detroit, Mich.), 0.5% glucose and 0.01% Sequestrene (Ciba Geigy, Basel, Switzerland) and sterilized by autoclaving (121° C., 20 min). Erlenmeyer flasks containing 100 ml of the same culture medium with 1 mg of cholesterol added, were inoculated with 10 ml of a late log phase culture and incubated for 24 hs, at 30° C., on a rotary shaker at 150 rpm. Cell densities were estimated by counting appropriate dilutions of the cultures fixed with 2% formaldehyde in a Neubauer hemocytometer.

Preparation of the homogenate fraction: the ciliates were harvested by centrifugation at 15,000 g for 10 min, re-suspended in 0.2 M Na₂HPO₄ buffer (pH 7.2), washed once in the same buffer and finally suspended in 10 mM Tris-HCl, pH 7.2, 250 mM sucrose, 5 mM MgCl₂, 1 mM EDTA (TES-MG buffer). The suspensions were homogenized by sonication (Vibracell, Sonics & Materials, Inc) in an ice-water bath until complete cell lysis (6 min, approximately). The suspension was centrifuged at 8,800× g for 10 min, and the supernatant (homogenate) was tested for desaturase activity.

Test for desaturase activity: cholesterol desaturase activity was assayed essentially as described for a fatty-acid desaturase from Tetrahymena termophila (Bertram J. and Erwin J. J. Protozool. 28:127-131, 1981). 5 ml of the homogenate (approximately 250 mg of protein) was incubated with 0.01 mg unlabeled cholesterol as the carrier and 0.5 Ci of [³H] cholesterol (46 Ci/mmol, Amersham Life Sciences, Buckinghamshire, U.K.). The mixture was incubated for 3 hours in a shaking water bath (150 rpm) at 30° C. Boiled homogenate preparations (10 min, 100° C.) were used as controls. The reaction was stopped by addition of one volume of 2 M sodium hydroxide prepared in methanol/water (1:1 v/v). The mixture was saponified during 1 hour at 60° C. and further extracted.

Analysis of sterols: the unsaponifiable fraction which includes the sterols, was extracted twice with the same volume of chloroform, evaporated under nitrogen at 60° C., suspended in 100 μl of chloroform and separated by HPLC using a C18 column. Methanol/ water (95:5 v/v) was used as mobile phase at 1.3 ml/min. Sterols detection and quantification was monitored with an U.V. detector at 205 nm and 285 nm, or with a radioactivity detector as indicated above (Radiomatic Camberra Instruments).

To aid quantitation, stigmasterol (100 μg/ml) was added to the reaction mixture prior to saponification and used as internal standard. This sterol separates cleanly from cholesterol and its derivatives under the conditions employed.

The extent of cholesterol desaturase activity was estimated from the conversion of the radiolabeled cholesterol added and was expressed as mmol of product formed/mg of protein x min.

### Example 2

### Preparation of the Microsomal Fraction

Preparation of a microsomal fraction from Tetrahymena termophila cells: The procedure for Tetrahymena cell culture and homogenate preparation was identical to that set in example 1. The cell free homogenate was further centrifuged at 105,000× g for 1.5 hours, and the resulting pellet was separated and suspended in the same buffer (TES-Mg buffer) to 5-10 mg/ml final protein concentration. All procedures were carried out at 4° C.

### Example 3

### Preparation of the Desaturase Enriched Fraction

Preparation of a desaturase enriched fraction from Tetrahymena termophila cells: The procedure for Tetrahymena cell culture and microsomal preparation was identical to that set in Examples 1 and 2. 50 ml of the microsomal fraction (5 mg/ml, protein concentration) was mixed with a detergent solution (N-Octyltioglucoside, Sigma Chem, 5% w/v) to get a final detergent concentration of 0.35% at pH 7.0. The detergent/protein ratio was between 0.05 and 10 (w/w).

The suspension was stirred during 4 hours at 15° C. and centrifuged at 105,000× g, 1.5 hours, to remove the non-solubilized components.

After ultracentrifugation and pellet separation solid (NH₄)₂SO₄ was added to the supernatant in a water bath set at 5° C. until the concentration of (NH₄)₂SO₄ reached 45% w/v. The precipitate formed was centrifuged 10 min at 10,000× g at 5° C. The supernatant was discarded and the precipitate was suspended in 2 ml of a phosphate buffer solution (0.1M, pH 7.2). This solution was dialyzed during 12 hours at 5° C. with gentle stirring in 500 ml of buffer containing 0.1 M phosphate pH 7.2, 250 mM Sucrose, 0.1 mM EDTA.

After 3 changes of this solution, the sample was diluted to a final protein concentration of 10 mg/ml. This procedure concentrated the cholesterol desaturase activity in a protein-enriched fraction which was tested for desaturase activity.

Test for desaturase activity: the procedure for assaying the cholesterol desaturase activity in desaturase- enriched fractions was essentially as described in example 1, except that 1.0 ml of the protein-enriched fraction (10.0 mg/ml protein) was used. This volume was incubated with 0.01 mg unlabeled cholesterol as the carrier and 0.5 Ci of [3H] cholesterol (46 Ci/mmol, Amersham Life Sciences, Buckinghamshire, U.K.). Pyridin cofactors (NAD, NADP, NADH and NADPH; 5 mM each one) were added and the mixture was incubated at 30° C. during 3 hours with gentle stirring (100 rpm). Sterols analysis, including saponification, extraction, detection and quantification were as described in example 1.

### Example 4

### Purification of 7 Cholesterol Dasaturase

Step 1: Culturing a microorganism in a suitable medium, wherein said microorganism is capable of producing 7 cholesterol desaturases. Tetrahymena thermophila wild type strain CU399 was maintained in axenic cultures. Inocula were prepared in medium containing 1% proteose peptone, 0.1% yeast extract (both from Difco Laboratories, Detroit, Mich.), 0.5% glucose and 0.01% Sequestrene (Ciba Geigy, Basel, Switzerland) and sterilized by autoclaving (121° C., 20 min). Erlenmeyer flasks containing 100 ml of the same culture medium with 0,5 mg of 22 dehydrocholesterol added, were inoculated with 10 ml of a late log phase culture and incubated for 24 hs, at 30° C., on a rotary shaker at 150 rpm.

Step 2: Desintegrating the culture and extracting same with a buffer solution. The ciliates were harvested by centrifugation at 15,000 g for 10 min, resuspended in 0.2 M Na₂HPO₄ buffer (pH 7.2), washed once in the same buffer and finally suspended in 10 mM Tris-HCl , pH 7.2, 250 mM sucrose, 5 mM MgCl₂, 1 mM EDTA and 2 uM of 2- mercaptoethanol (TES-Mg buffer). The suspensions were homogeneized by sonication (Vibracell, Sonics & Materials, Inc) in an ice-water bath until complete cell lysis (6 min, approximately). The suspension was centrifuged at 8,800× g for 10 min, and the supernatant (homogenate) was further centrifuged at 105,000× g for 1.5 hours, and the resulting pellet (microsomal fraction) was separated and suspended in the same buffer (TES-Mg buffer) to 5-10 mg/ml final protein concentration.

This suspension was mixed with a detergent solution (N-Octyltioglucoside,Sigma Chem, 5% w/v) to get a final detergent concentration of 0.35% at pH 7.0. The detergent/protein ratio was between 0.05 and 10 (w/w).

The suspension was stirred during 4 hours at 15° C. and centrifuged at 105,000×g, 1.5 hours, to remove the non-solubilized components. The supernatant is a solubilized protein solution

Step 3: Recovering said cholesterol Desaturase Therefrom.

The solubilized fraction is concentrated to a small volume by 60% saturation with ammonium sulfate precipitation.

After centrifugation, the precipitated protein is dissolved in 50 mM sodium phosphate buffer (pH 7.4 and containing 10% of glycerol and 2 uM of 2-mercaptoethanol) to give a protein concentration of 10 mg/ml.

The solution is then applied to a Phenyl sepharose column (Amersham Pharmacia Biotech) that has been equilibrated with the same buffer. The column is washed out with 50 mM sodium phosphate buffer (pH 7.4 and containing 10% of glycerol and 2 uM of 2-mercaptoethanol) plus 1.0 M NaCl and then with the same buffer but without the addition of salt.

The eluted protein fraction was applied to a cation exchange chromatography (SP HiTrap column, Amersham Pharmacia Biotech) filled with 1.0 ml of SP Sepharose High performance (Pharmacia Biotech) equilibrated with 50 mM sodium phosphate buffer (pH 7.4).

After washing with 2 column volumes (2.0 ml) of the same buffer the enzyme was eluted by a linear NaCl gradient (0 to 1.0 M NaCl in 10 column volumes) in the same buffer (plus 10% glycerol)at a flow rate of 1.0 ml/min.

2 ml fraction were collected and the desaturase activity was tested.

Fraction eluted between 200-400 mM of NaCl show maximal desaturase activity.

This fraction is applied to a Superose 12 FPLC column (HR 10-30) equilibrated with 50 mM sodium phosphate buffer (pH 7.4 and containing 10% of glycerol and 2 uM of 2-mercaptoethanol). Activity fractions are eluted with the same buffer at a flow rate of 0.5 ml/min. the active fraction are collected and stored.

### Example 5

### Purification of 22 Cholesterol Desaturase

Step 1: culturing a microorganism in a suitable medium, wherein said microorganism is capable of producing 22 cholesterol desaturases. Tetrahymena thermophila wild type strain CU399 was maintained in axenic cultures. Inocula were prepared in medium containing 1% proteose peptone, 0.1% yeast extract (both from Difco Laboratories, Detroit, Mich.), 0.5% glucose and 0.01% Sequestrene (Ciba Geigy, Basel, Switzerland) and sterilized by autoclaving (121° C., 20 min). Erlenmeyer flasks containing 100 ml of the same culture medium with 1.0 mg of cholesterol added, were inoculated with 10 ml of a late log phase culture and incubated for 24 hs, at 30° C., on a rotary shaker at 150 rpm.

Step 2: Desintegrating the Culture and Extracting Same with a Buffer Solution.

The ciliates were harvested by centrifugation at 15,000 g for 10 min, resuspended in 0.2 M Na₂HPO₄ buffer (pH 7.2), washed once in the same buffer and finally suspended in 10 mM Tris-HC1, pH 7.2, 250 mM sucrose, 5 mM MgCl₂, 1 mM EDTA (TES-Mg buffer). The suspensions were homogeneized by sonication (Vibracell, Sonics & Materials, Inc) in an ice-water bath until complete cell lysis (6 min, approximately). The suspension was centrifuged at 8,800× g for 10 min, and the supernatant (homogenate) was further centrifuged at 105,000× g for 1.5 hours, and the resulting pellet (microsomal fraction )was separated and suspended in the same buffer (TES-Mg buffer) to 5-10 mg/ml final protein concentration.

This suspension was mixed with a detergent solution (N-Octyltioglucoside, Sigma Chem, 5% w/v) to get a final detergent concentration of 0.35% at pH 7.0. The detergent/ protein ratio was between 0.05 and 10 (w/w).

The suspension was stirred during 4 hours at 15° C. and centrifuged at 105,000×g, 1.5 hours, to remove the non-solubilized components. The supernatant is a solubilized protein solution

Step 3: Recovering Said Cholesterol Desaturase Thereform.

The solubilized fraction obtained as in example 3 is concentrated to a small volume by 60% saturation with ammonium sulfate precipitation.

After centrifugation, the precipitated protein is dissolved in 50 mM sodium phosphate buffer (pH 7.4 and containing 10% of glycerol) to give a protein concentration of 10 mg/ml.

The solution thus obtained is applied to a column (XK16/20, Pharmacia Biotech) filled with 20 ml of Q Source 30 (Pharmacia Biotech) equilibrated with buffer 50 mM sodium phosphate buffer (pH 7.4 and containing 10% of glycerol). After washing with 2 column volumes of the same buffer, a linear NaCl gradient (0 to 1.0 M, in 15 column volumes of the same buffer) was applied for the elution at a flow rate of 0.9 ml/min. 20 ml fractions were collected to test the Δ22 desaturase activity in 2 ml samples (with the addition of radiolabeled cholesterol) Fraction between 5-15% of buffer B (50-150 mM of NaCl) showed maximal Δ22 desaturase activity.

The active fraction is collected, pooled and stored.

This active pool is submitted to gel filtration through a column of Superose 12 (HR 10/30, Pharmacia Biotech) and developed with buffer 50 mM sodium phosphate (pH 7.4 and containing 150 mM NaCl). Active fractions were eluted with the same buffer at a flow rate of 0.5 ml/min and collected for the assay of Δ22 desaturase activity.

### Example 6

### Production of Substantially Pure 7 Dehydrocholesterol (Provitamin D₃) from Cholesterol

A substrate solution was prepared by dissolving pure cholesterol in hot ethanol to a concentration of 5 mM.

A substantial pure enzyme solution was prepared as was described in example 4.

0.9 ml of enzyme solution were mixed with 0.1 ml of substrate solution and incubated at 30° C.

After 3 hours of incubation in the same condition the reaction was stopped by addition of one volume of 2 M sodium hydroxide prepared in methanol/water (1:1 v/v). The mixture was saponified during 1 hour at 60° C. and further extracted with hexane.

The mixture of sterols (Delta 7 dehydrocholesterol and cholesterol) present in the unsaponifiable fraction was acetilated and the mixture was separated into its components by silver nitrate column chromatography.

The column was eluted with a gradient of benzene-petroleum ether and the fraction were monitored by UV absorbance and HPLC analysis.

Pure fraction was collected, pooled and crystallized.

### Example 7

### Production of Substantially Pure 7,22 Di Dehydrocholesterol from Cholesterol

A substrate solution was prepared by dissolving pure cholesterol in hot ethanol to a concentration of 5 mM.

A substantial pure enzyme solution containing 7 desaturase was prepared as was described in example 4 (enzyme solution A)

A substantial pure enzyme solution containing 22 desaturase was prepared as was described in example 5 (enzyme solution B)

0.9 ml of enzyme solution A were mixed with 0.9 ml of enzyme solution B and 0.2 ml of substrate solution and incubated at 30° C.

After 1 hour of incubation in the same condition the reaction was stopped by addition of one volume of 2 M sodium hydroxide prepared in methanol/water (1:1 v/v). Sterols analysis, including saponification, extraction, detection and quantification were as described in example 6.

I claim:

1. A cell free extract from Ciliate phylum microorganism, wherein said cell free extract contains cholesterol desaturase activities selected from the group comprising Δ-7 and Δ-22 cholesterol desaturases activities that catalyze desaturation of cholesterol.

2. A cell free extract of claim 1, wherein said cell free extract is selected from the group consisting of cell free homogenate, microsomal fraction and desaturase-enriched fraction, or a combination thereof, all from Ciliata phylum microorganism.

3. A cell free extract of claim 1, wherein the ciliate is selected from the group consisting of Paremecium, Tetrahymena and Colpidium.

4. A process for manufacturing Δ7 dehydrochlesterol (provitamin D3) and Δ7,22 bis dehydrocholesterol comprising:
(a) mixing a cell free extract of claim 1 with a cholesterol substrate;
(b) incubating the mixture for a period of time enough to produce Δ7 dehydrocholesterol and Δ7,22 bis dehydrocholesterol;
(c) recovering said Δ7 dehydrocholesterol and Δ7,22 bis dehydrocholesterol by solvent extraction and chromatographic purification.

5. A substantial pure Δ7 cholesterol desaturase enzyme from Ciliata phylum microorganism, wherein said enzyme is capable of catalyzing the conversion of a cholesterol substrate in Δ7 dehydrocholesterol by introducing a double bound at the position seven in the cholesterol molecule.

6. A substantial pure Δ7 cholesterol desaturase enzyme of claim 5, wherein the ciliate is selected from the group consisting of Paremecium, Tetrahymena and Colpidium.

7. A substantial pure Δ7 cholesterol desaturase enzyme according to claim 5, the enzyme
(a) having a molecular weight of approximately 60 kDa by gel chromatography;
(b) having an optimum pH range for enzymatic activity between 6.5-8.5;
(c) having an optimum temperature range for enzymatic activity of 28° C. to 35° C.;
(d) being unaffected by metal ions such as Ca⁺², Mn⁺² and Mg⁺², EDTA concentrations and 2-mercaptoethanol;
(e) being inactivated after 1 minute at 100° C.;
(f) being storage at −20° C. by at least 6 months.

8. A substantial pure Δ22 cholesterol desaturase enzyme from Ciliata phylum microorganism, wherein said enzyme is capable of catalyzing the conversion of a cholesterol substrate in Δ22 dehydrocholesterol by introducing a double bound at the position twenty-two in the cholesterol molecule.

9. A substantial pure Δ22 cholesterol desaturase enzyme of claim 8, wherein the ciliate is selected from the group consisting of Paremecium, Tetrahymena and Colpidium.

10. A substantial pure Δ22 cholesterol desaturase enzyme according to claim 8, the enzyme
(a) having a molecular weight of approximately 60 kDa by gel chromatography;
(b) having an optimum pH range for enzymatic activity between 5.5-8.5;
(c) having an optimum temperature range for enzymatic activity of 28° C. to 35° C.;
(d) being unaffected by metal ions such as Ca⁺, Mn⁺² and Mg⁺² and EDTA concentrations;
(e) being inactivated after 1 minute at 100° C.;
(f) being storage at −20° C. by at least 6 months.

11. A process for preparing a substantial pure Δ7 cholesterol desaturase enzyme from Ciliata phylum microorganism according to claim 5, the process comprising the steps of:
(a) culturing a microorganism in a suitable medium, wherein said microorganism is capable of producing Δ7 cholesterol desaturases;
(b) disintegrating the culture and extracting the same with buffer solution containing, if necessary, non ionic surfactant or stabilizer as glycerol;
(c) subjecting the extract to a chromatography purification under suitable conditions; and
(d) eluting and recovering said Δ7 cholesterol desaturases.

12. The process according the claim 11, wherein the step of culturing is carried out in a medium containing 1% proteose peptone, 0.1% yeast extract, 0.5% glucose, 0.01% Sequestrene and 0,5mg % of 22 dehydrocholesterol.

13. The process according the claim 11, wherein the chromatography purification is selected from a group comprising size exclusion chromatography, anion exchange chromatography, cation exchange chromatography and combinations thereof.

14. A process for preparing a substantial pure Δ22 cholesterol desaturase enzyme from Ciliata phylum microorganism according to claim 8, the process comprising the steps of:
(a) culturing a microorganism in a suitable medium, wherein said microorganism is capable of producing Δ22 cholesterol desaturases;
(b) disintegrating the culture and extracting the same with buffer solution containing, if necessary, non ionic surfactant or stabilizer as glycerol;
(c) subjecting the extract to a chromatography purification on a suitable chromatography conditions; and
(d) eluting and recovering said Δ22 cholesterol desaturases.

15. The process according the claim 14, wherein the step of culturing is carried out in a medium containing 1% proteose peptone, 0.1% yeast extract, 0.5% glucose, 0.01% Sequestrene and 1.0 mg % of cholesterol.

16. The process according the claim 14, wherein the chromatography purification is selected from a group comprising size exclusion chromatography, anion exchange chromatography, cation exchange chromatography and combinations thereof.

17. The use of substantial pure Δ7 cholesterol desaturase enzyme from Ciliata phylum microorganism of claim 5 for producing Δ7 dehydrocholesterol (provitamin D3) employing cholesterol as substrate.

18. The use according the claim 17, wherein the cholesterol substrate es seleccionado del grupo comprendido por colesterol puro, cholesterol-containing products and cholesterol enriched fractions.

19. The use according the claim 17, wherein the ciliate is selected from the group consisting of Paremecium, Tetrahymena and Colpidium.

20. The use of pure Δ7 cholesterol desaturase and substantial pure Δ22 cholesterol desaturase enzymes from Ciliata phylum microorganism of claims 5 and 8 for producing Δ7,22 bis dehydrocholesterol employing cholesterol as substrate.

21. The use according the claim 20, wherein the cholesterol substrate es seleccionado del grupo comprendido por colesterol puro, cholesterol-containing products and cholesterol enriched fractions.

22. The use according the claim 20, wherein the ciliate is selected from the group consisting of Paremecium, Tetrahymena and Colpidium.
